(19) United States
(12) Patent Application Publication
(10) Pub. No.: US 2024/0207636 A1
(43) Pub. Date: Jun. 27, 2024

(54) DEVICE FOR STIMULATING THE BIOSYNTHESIS OF VITAMIN D3

(71) Applicant: JK-HOLDING GMBH, Windhagen (DE)

(72) Inventor: Jürgen GERSTENMEIER, Neuwied (DE)

(73) Assignee: JK-HOLDING GMBH, Windhagen (DE)

(21) Appl. No.: 18/555,998

(22) PCT Filed: Apr. 19, 2022
(86) PCT No.: PCT/IB2022/053653

§ 371 Date: Oct. 18, 2023

(30) Foreign Application Priority Data
Apr. 21, 2021 (CH) 00425/21

(51) Int. Cl. A61N 5/06

(52) U.S. Cl. CPC A61N 5/062; A61N 5/0616; A61N 2005/0626; A61N 2005/066; A61N 2005/0661; A61N 2005/0663; A61N 2005/0664

(57) ABSTRACT

The present invention relates to a device (1) for stimulating the biosynthesis of vitamin D3. The device comprises at least one first light source for emitting light radiation comprising wavelengths in a range of 280 nm to 315 nm. It further comprises a sensor unit for detecting an irradiation area, and a directing unit for aligning the at least one light source with the irradiation area. The device also comprises a control unit, which is designed to identify the irradiation area and to trigger an emission of light radiation comprising wavelengths in a range of 280 nm to 315 nm by the at least one light source.

The present invention further comprises a computer program product for controlling such a device.

The present invention relates to a device for stimulating the biosynthesis of vitamin D3 and a computer program product for controlling the same, all in accordance with the preambles of the independent claims.

### TECHNOLOGICAL BACKGROUND

Vitamins are essential substances for an organism, which take part in numerous metabolic reactions and have an influence on the immune system. Among the vitamins, vitamin D plays a special role. Vitamin D is a lipophilic compound that can be synthesized by most vertebrates by exposure of the skin to ultraviolet light. The main reason for this is the UV-B component in sunlight, i.e. light radiation with wavelengths of 280 nm to 315 nm.

Vitamin D3 supplementation may be necessary at various latitudes or when engaging in activities that are primarily indoors or at night. In addition to foods containing vitamin D, vitamin D can also be taken as a dietary supplement. It is important that when taking vitamin D tablets, a corresponding fat or oil is also taken to facilitate absorption. Basically, in environments in which it is not possible to keep the vitamin D content at a sufficient level through daily exposure to a certain amount of sunlight or through appropriate supplements, it is possible to utilize biosynthesis via the skin through light irradiation, as would be done with exposure to sunlight. For example, visits to the solarium with a short exposure time can be sufficient to keep the body's own biosynthesis of vitamin D3 at a sufficient level. Products are increasingly appearing on the market that advertise light sources for daily use that are supposed to additionally support the biosynthesis of vitamin D3. However, it is important to note that the relevant UV radiation in the wavelength range of 280 nm to 315 nm can damage the eyes. Type A and B UV radiation is usually absorbed by the lens of the eye, so over time clouding effects can occur and result in cataracts that require surgical treatment. In addition, there is a certain risk of cancer from said UV radiation, so it is preferable to control the exposure and not to carry it out for long periods of time.

Above all, professional groups that regularly have to work at night or people who live in places where the daily solar radiation is very low or absent, especially in the winter months, benefit greatly from light sources that enable the body's own and natural biosynthesis of vitamin D3. In the pandemic year 2020, a scientific study also found strong evidence that UV-B radiation can have a protective effect against a severe course of Covid-19, supported by vitamin D3 synthesis (Evidence of protective role of Ultraviolet-B (UVB) radiation in reducing COVID-19 deaths [Moozhipurath, R. K., Kraft, L. and Skiera, B., Sci Rep 10, 17705 [2020]).

For the purposes of the present invention, and not wishing to be bound by this theory, a biosynthesis of vitamin D3 and its corresponding derivatives is assumed for the irradiation, taking place with ultraviolet light in the wavelengths of 290 to 315 nm, i.e. wavelengths in the UV B range with an energy of at least half the minimum erythema dose (MED). For this purpose, 7-dehydrocholesterol is broken down by a photochemically induced reaction of the B ring and previtamin D3 is formed. This previtamin D3 is unstable and reacts to form vitamin D3. In the blood, it is bound to the vitamin D-binding protein and transported to the liver, where it is hydroxylated to form calcitriol. The required precursor, i.e. 7-dehydrocholesterol, is produced by the body itself.

In summary, there is a need for devices that can stimulate or at least support the body's own biosynthesis of vitamin D3. These devices should have as few harmful side effects as possible when used.

### PRESENTATION OF THE INVENTION

It is therefore an object of the present invention to provide a device of the type mentioned at the beginning which overcomes at least one disadvantage of the known art.

In particular, such a device is to be provided which is safe to use and can have a measurable effect on the vitamin D3 content in the blood. A further object of the present invention is to provide a computer program product which is suitable for controlling such a device and enables safe operation of such a device.

It is a further particular object of the present invention to provide such a device and a corresponding computer program product which inhibits or substantially prevents damage to the lens of the eye as a result of its use.

At least one of these objects has been solved with a device and a computer program product according to the characterizing part of the independent claims.

One aspect of the present invention relates to a device for stimulating the biosynthesis of vitamin D3. The device comprises at least one first light source for emitting light radiation comprising wavelengths in a range of 280 nm to 315 nm. The device further comprises a sensor unit for detecting an irradiation area. It further comprises a directing unit for aligning the at least one light source with the irradiation area. In addition, the device according to the invention comprises a control unit which is designed to identify the irradiation area and to trigger an emission of light radiation comprising wavelengths in a range of 280 to 315 nm by the at least one light source.

An advantage of the device according to the invention can be that the sensor unit can be used to ensure that the emission of light radiation is only utilized when an irradiation area is detected that is safe for the irradiation. This irradiation area can be determined using predefined criteria, for example.

It was surprisingly found that, using a device according to the invention, an irradiation area, for example an area of human skin, can be irradiated with sufficient light radiation comprising wavelengths in particular in the UV-B range in order to stimulate or at least support the body's own formation of vitamin D and thereby to remain below a minimum erythema dose for a fair-skinned human.

For the purposes of the present invention, the minimum erythema dose is to be understood as the threshold value above which an erythema, that is to say a sunburn effect, can be observed on the skin. The minimum erythema dose is different for different skin types, and is also subject to individual variations. For the present invention, for example, it can be assumed as a reference that an irradiation dose of about 12.5 mJ/cm² for a period of 10 to 15 minutes is sufficient for a person with skin type 2 to stimulate biosynthesis of vitamin D3. For the purposes of the present invention, the skin type can be understood as being divided according to Fitzpatrick into the following types: I (very light), Il (light), III (medium light), IV (brownish, olive-colored), V (light brown, dark) and VI (dark brown to black) [T. B. Fitzpatrick: Ultraviolet-induced pigmentary changes: Benefits and hazards. In: Therapeutic Photomedicine. (=Current Problems in Dermatology. Volume 15). Karger, 1986, pp. 25-38].

In a particular embodiment, the light source comprises at least one LED. For the purposes of the present invention, a light source for emitting light radiation comprising wavelengths in a range of 280 nm to 315 nm is to be understood as a light source which covers at least a portion of this spectrum. Also suitable are, for example, UV-LEDs with a spectrum of 280 to 315 nm, as are LEDs which have a peak in said spectral range. Suitable light source can be determined by a person skilled in the art.

LEDs offer another advantage: it was surprisingly found in particular that LEDs can be used to stimulate the biosynthesis of vitamin D3 even more efficiently than natural sunlight. (Kalajian, T. A., Aldoukhi, A., Veronikis, A. J. et al. Ultraviolet B Light Emitting Diodes (LEDs) Are More Efficient and Effective in Producing Vitamin D3 in Human Skin Compared to Natural Sunlight. Sci Rep 7, 11489 (2017)).

The light source is particularly preferably designed to emit light radiation comprising wavelengths in a range of 290 nm to 300 nm.

For the purposes of the present invention, a directing unit can be understood to mean, for example, a mechanical arrangement which is suitable for directing the at least one first light source and/or at least the light radiation of the at least one first light source onto an irradiation area. This can be accomplished, for example, in that a directing unit can be moved and/or pivoted on at least one axis. The directing unit is preferably movable and/or pivotable on at least two axes, so that preferably a cone area, starting from the at least one first light source, can be traversed by the directing unit, so that a plurality of irradiation areas can be sensed within this cone area.

In a particular embodiment, the directing unit is designed to align the at least one light source in an angular range of 180°.

In a particular embodiment, the control unit comprises at least one processor that is designed to execute a computer program that is suitable for evaluating data received from the sensor unit. This evaluation is designed, for example, to identify a specific irradiation area. The control unit can be designed to carry out corresponding processes once an irradiation area has been identified. These processes can comprise, for example, emission of light radiation of said wavelengths, or switching off emission of light radiation of said wavelengths. Furthermore, these processes can be designed to cause alignment by the directing unit. If an irradiation area is detected continuously, the control unit can be designed, for example, to give the directing unit instructions to follow a specific course and to find a suitable irradiation area within a cone, for example, which the directing unit can detect. Once such an area has been identified, the control unit can then trigger an emission of light radiation of the said wavelengths.

In a particular embodiment, the sensor unit comprises an optical sensor. In particular, the optical sensor is a camera. In principle, all sensor elements that are capable of detecting an irradiation area over a certain distance are suitable for a sensor unit according to the invention. It has been shown that optical sensors are particularly suitable. The sensor unit can specifically comprise a sensor, e. g. a camera. It can also comprise other sensors, such as a range finder. Suitable range finders can be provided, for example, based on laser range finders, which are capable of emitting a laser beam and thereby accurately measure a distance to an object. This has the advantage, in particular, that the apparatus can use the controller to precisely determine an intensity for the light source, which takes into account the distance of the irradiation area from the light source. In this way, optimal stimulation of the biosynthesis of vitamin D3 can be made possible and it can be ensured that light radiation is applied in such a way that it remains below the critical erythema dose.

The optical sensor can also be designed to detect infrared images. A sensor equipped in such a way can, for example, track the effect of light radiation on an irradiation area. This can further support compliance with the minimum erythema dose. In addition, the thermal gradients can be used to determine a specific energy value that was applied to the irradiation area. From this value, for example, the administration of a sufficient dose of the light radiation mentioned can then be calculated.

In a particular embodiment, the device comprises a plurality of sensor units, each with one or more sensors, in particular optical sensors.

In a particular embodiment, the control unit is designed to control an alignment of the first light source by the directing unit based on an identified irradiation area. In this embodiment, for example, a sensor unit can be equipped with an optical sensor that covers a particular area. The range can be broad using a wide-angle lens. If a suitable irradiation area is recognized by the sensor unit within this area and identified by the control unit, the control unit can, for example, cause the directing unit to align the light source to this area. For this purpose, the control unit can be designed to define the irradiation area in the image area of the optical sensor based on a vector and to cause the directing unit to align the light source based on this vector.

In a particular embodiment, the device according to the invention comprises a collecting unit for generating a directed bundle of rays from the emission of light radiation by the first light source. The collecting unit is preferably designed to apply the light radiation to a limited irradiation area. For this purpose, the collecting unit has, for example, elements which make it possible to keep the light radiation within a certain space over the distance from the irradiation area, so that the irradiation area is defined as precisely as possible.

In a particular embodiment, the collecting unit comprises at least one reflector. For example, an LED can be used as the light source and a corresponding reflector arranged around the LED can be provided, which directs the light radiation emitted by the LED onto a specific irradiation area. A reflector unit which is adjustable is particularly preferably provided. An adjustable reflector unit can be made possible, for example, with a plurality of reflector planes that can be activated, with which, for example, a specific distance or a specific angle of the irradiation area relative to the light source can be taken into account.

In a particular embodiment, the collecting unit comprises a lens and/or a collimator. The collecting unit particularly preferably comprises a collimator with a collecting lens for generating a directed bundle of rays from the emission of light radiation by the first light source. Depending on the configuration of the apparatus, final size and placement relative to the irradiation area, the collecting unit can be designed differently by a person skilled in the art if required.

In a particular embodiment, the device according to the invention comprises a second light source for emitting light radiation comprising wavelengths in the visible range. The second light source is particularly preferably designed to emit light radiation comprising wavelengths in a range of 380 nm to 780 nm. By emitting visible light, the operation of the device according to the invention can ideally be followed by a human. Thus, this second light source can preferably be designed to irradiate the same irradiation area as the first light source. The generated “light cone” shows the user that a specific irradiation area is currently being exposed to the corresponding light radiation. For example, the user can be encouraged to keep the area still for the duration of the irradiation, thus ensuring that the apparatus can emit the defined and optimal amount of radiation to optimally carry out the intended stimulation of the biosynthesis of vitamin D3.

In a particular embodiment, the device according to the invention comprises a third light source for emitting light radiation comprising wavelengths in the near infrared range.

These wavelengths are particularly preferably in a range of 640 nm to 1,800 nm. Without wishing to be bound by this theory, exposure of the irradiation area to radiation in the near infrared range can improve blood flow in this area. Near-infrared radiation is employed to increase blood flow to the deeper layers of the tissue. As a result, biosynthesis of vitamin D3 can be stimulated even more, which in turn makes it possible to further reduce the dose of UV-B radiation.

In a particular embodiment, the control unit is designed to identify a human face in an irradiation area. This can be done, for example, by comparing an irradiation area recognized by a sensor unit with a database. Methods for face recognition are now known and are widely used. Modern cameras, such as those built into current mobile phones, for example, have face recognition to support the corresponding sharpness of an image. Face recognition, which recognizes the presence of a face and cannot necessarily assign the face to a specific person, is sufficient for the purposes of the present invention. Nevertheless, it would be conceivable to use facial recognition to identify a specific person. This can make it possible, for example, to use the device according to the invention to deliver a certain dose to a person over a certain period of time. The apparatus can thus determine that the person in question has been sufficiently exposed to the light radiation within a certain period of time in order to stimulate the corresponding biosynthesis of vitamin D3. Two-dimensional and three-dimensional face recognition methods that are suitable for use in the present invention are known to the person skilled in the art and can be adapted if necessary to the requirements, e. g. depending on whether a mere recognition, whether a face is there, or assignment of a face to a specific person is desired.

In a particular embodiment, the control unit is designed to recognize the face of a minor and to exclude this person from the radiation. In a particular embodiment, the control unit is designed to match a recognized face with a register that is approved for the radiation. It can thus be ensured that only approved and/or authorized users use the device according to the invention.

In a particular embodiment, the control unit is designed to identify a predefined irradiation area. For example, an irradiation area that has already been irradiated can be considered a predefined irradiation area, so that the control unit is able to identify an already irradiated irradiation area and decides to use a different irradiation area for a second irradiation within a specific time interval. It can thus be ensured that a daily dose of UV-B radiation is not exceeded in a specific irradiation area. It would also be conceivable for the control unit to be designed to recognize predefined irradiation areas that are excluded from irradiation. For example, irradiation areas of the skin, which contain scar tissue, wound tissue, scabs, tattoos, skin changes such as nevi and the like, may be excluded from the irradiation. Such predefined irradiation areas can be stored in a memory, or they can be calculated from the identified irradiation area, similar to what happens in face recognition, by detecting individual objects two-dimensionally or three-dimensionally and then identifying them using algorithmic methods.

In a particular embodiment, the device according to the invention comprises a communication unit for exchanging data. The communication unit particularly preferably comprises at least one interface for data transmission with a computer apparatus, which can also include a modern cell phone. This interface can be suitable for enabling wireless data transmission, for example using a Wi-Fi protocol or a Bluetooth® protocol. However, the interface can also be suitable for ensuring data transmission by means of a plug and cable connection based on a USB protocol. A communication unit makes it possible, for example, to monitor or configure an evaluation of an operation by the device according to the invention on a third-party apparatus. For example, use can provide for certain user data, such as, for example, skin type, age, occupation and lifestyle, to be stored with a profile. The apparatus can be designed to ensure optimal support for the biosynthesis of vitamin D3 on the basis of this data. The communication unit can also be designed, for example, to keep programming in the form of firmware of the device up to date. It is also conceivable that the communication unit enables a connection to a machine-learning-supported program, as a result of which face recognition, for example, can be continuously improved. Further functions would then be, for example, the recognition of other user areas excluded from radiation, such as birthmarks or wounds, as already described above.

In a particular embodiment, the first light source and/or the second light source and/or the third light source are physically coupled around the sensor unit. Thus, they can be jointly directed onto the irradiation area by the directing unit. This arrangement is particularly advantageous if the device according to the invention is to have a “search and find” function in which it actively identifies a suitable irradiation area. For example, a movement sensor can be provided in the sensor unit, which recognizes that a person is in an effective range of the device according to the invention. The sensor unit can then begin to detect specific irradiation areas and to identify them using the control unit. The control unit can cause a directing unit to search for a suitable irradiation area within the effective area of the device until such an area is found.

In a particular embodiment, the directing unit is designed to be pivotable about at least two axes. Said directing unit particularly preferably has at least one rotatably mounted stand base and a rotatably mounted tool arm for pivoting the light source on at least two axes. Such a configuration makes it possible to expand the effective range of the device according to the invention to a hemisphere on the plane on which the device is placed. Due to the movement on two axes, the entire space inside this main sphere can be detected by the sensor unit.

In a particular embodiment, the irradiation area is a skin area with an area of 40 cm² to 900 cm², in particular 50 cm² to 600 cm², particularly preferably of approximately 400 cm². A person skilled in the art can find further suitable irradiation protocols in the study by Kalajian, T. A., Aldoukhi, A., Veronikis, A. J. et al. and Moozhipurath, R. K. et al, supra.

In a particular embodiment, the skin area of this irradiation area can be defined by a regulated collecting unit. For this purpose, the collecting unit can have an adjustable aperture, for example, which can widen and narrow the cone of the light beam accordingly, so that the irradiation area can be defined within certain parameters.

With the device according to the invention, a device is provided which makes it possible to stimulate the biosynthesis of vitamin D3 in a targeted manner and with good control of possible side effects. A person skilled in the art appreciates that in an embodiment of a device of this type according to the invention, any combination of the features mentioned can be implemented, provided they are not mutually exclusive.

Another aspect of the present invention relates to a computer program product for controlling a device for stimulating biosynthesis of vitamin D3. The computer program product is particularly preferably executed by a control unit of the device described above. The computer program is designed to compare an irradiation area recognized by a sensor unit with predefined irradiation areas. Said computer program is further designed to trigger an emission of light radiation comprising wavelengths in a range of 280 nm to 315 nm by at least one first light source. This occurs when the recognized irradiation area does not correspond to an irradiation area excluded from exposure.

The computer program product according to the invention can thus ensure that the irradiation can always meet health requirements. The suitable irradiation areas are particularly preferably predefinable.

In a particular embodiment, an irradiation area excluded from irradiation is an irradiation area selected from the group consisting of: a human face, a skin area provided with one or more pigmented nevi, a skin area provided with scab, wound and/or scar tissue, and a skin area already irradiated with light radiation comprising wavelengths in a range of 280 nm to 315 nm within a predefined time interval.

In a particular embodiment, an irradiation area excluded from irradiation is an irradiation area that is part of a person who is not authorized to use the device. This can be ensured, for example, by registering and storing a facial image as a biometric identification means. In addition, minors can be excluded from using the device in this way.

In a particular embodiment, the computer program product is designed to assign a time interval to a specific irradiation area. Within this time interval, the irradiation area is to be irradiated with light radiation comprising wavelengths in a range of 280 to 315 nm. This can be done, for example, in cooperation with the device mentioned, in that the control unit identifies a specific irradiation area and recognizes it as suitable. The irradiation is initiated and a certain time interval is established in which the irradiation is to take place. This time interval can be designed to be fixed, for example a time interval of between half a minute and ten minutes, in particular between one and eight minutes, more particularly between three and five minutes, or it can be variable and defined using parameters which, for example, have been previously stored in a user profile, comprising skin type, vitamin D requirement, lifestyle, etc. It is known, for example, that the darker the skin type, the longer the person has to be in the sun before a sufficient amount of vitamin D3 can be synthesized. Furthermore, a lifestyle that, for example, takes place mainly during the night hours or takes place beyond a latitude at which daily solar radiation falls below a certain level, can also be a reason to adjust the corresponding time intervals. A medical history can also be a reason to make an appropriate adjustment, such as, for example, a disorder of the vitamin D metabolism or a special need for vitamin D due to age and/or medical conditions.

In a particular embodiment, the computer program product is designed to control a search for a predefined irradiation area by the computer program product controlling the sensor unit. If no suitable irradiation area is found, the sensor unit can be designed to carry out a search by scanning the entire effective area and identifying a possible irradiation area.

In a particular embodiment, the computer program product is designed to compare each image recorded by an optical sensor with an irradiation area that is excluded from irradiation.

In this way, it can be ensured that an irradiation area that is not intended for exposure is not accidentally exposed.

In a particular embodiment, the computer program product is designed to enter into an operating mode by a signal from the sensor unit, in particular to search for a suitable irradiation area that is not excluded from irradiation. This signal can be a motion detection, for example.

In a further particular embodiment, the computer program product is designed to cause stopping irradiating an irradiation area with light radiation comprising the wavelengths in a range of 280 to 315 nm as soon as a sensor unit detects an irradiation area excluded from irradiation in the effective range of the light source. This can happen, for example, if an irradiated person moves or another person enters this irradiation room, so that it can no longer be ensured that an area excluded from the irradiation, such as a face, will not be affected by the radiation.

With the device according to the invention and the corresponding computer program product, a means is provided to provide a device for stimulating the biosynthesis of vitamin D3 that can be used in many areas. The device shown is safe to use and prevents many negative side effects of exposure to UV-B radiation, i.e. radiation in a wavelength range of 280 to 315 nm.

The device according to the invention is now explained in more detail below using specific exemplary embodiments and figures, but without being bound by them. However, further advantageous embodiments of the device according to the invention result from these specific exemplary embodiments for the person skilled in the art.

In particular, the figures illustrate an implementation in a schematic way, in which reference numerals indicate the same element in each case with the same reference numeral for easier tracing.

### DESCRIPTION OF THE FIGURES

Exemplary embodiments of the invention are described with reference to the following figures.

FIG. 1a shows a device according to the invention for stimulating the biosynthesis of vitamin D3.

FIG. 1b shows the device of FIG. 1a in a frontal view;

FIG. 1c shows a cross section through the device of FIG. 1b in the C-C plane;

FIG. 1d shows a side view of the device of FIG. 1b;

FIG. 1e shows a side cross section of the device of FIG. 1b in the A-A plane;

FIG. 1f shows a side cross section of the device of FIG. 1b in the B-B plane;

FIG. 1g shows an exploded view of the device of FIG. 1a;

FIG. 1h shows a detailed view of a light source of FIG. 1a;

FIG. 1i shows an alternative arrangement of a light source for a device according to FIG. 1a;

FIG. 2 shows an alternative embodiment of a device according to the invention;

FIG. 3a shows another alternative embodiment of a device according to the invention in a front view, and

FIG. 3b shows the device according to the invention according to FIG. 3a in an isometric view.

### IMPLEMENTATION OF THE INVENTION

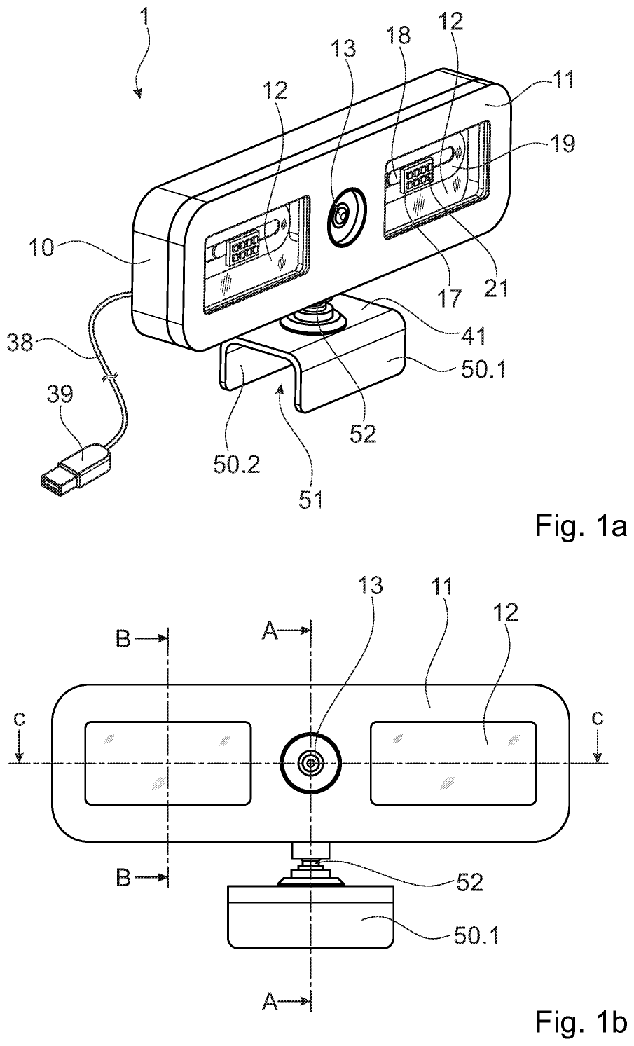

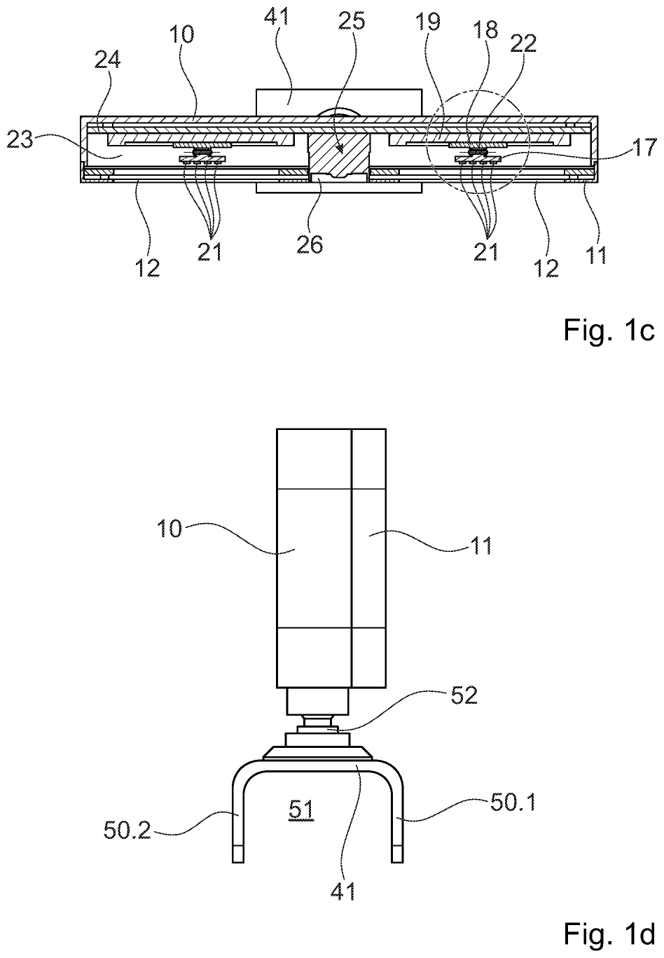

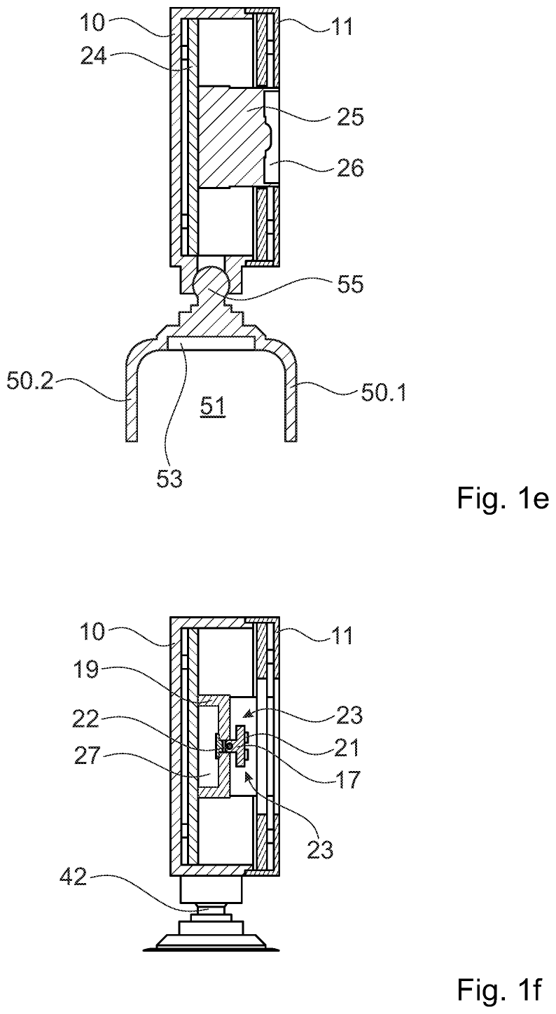

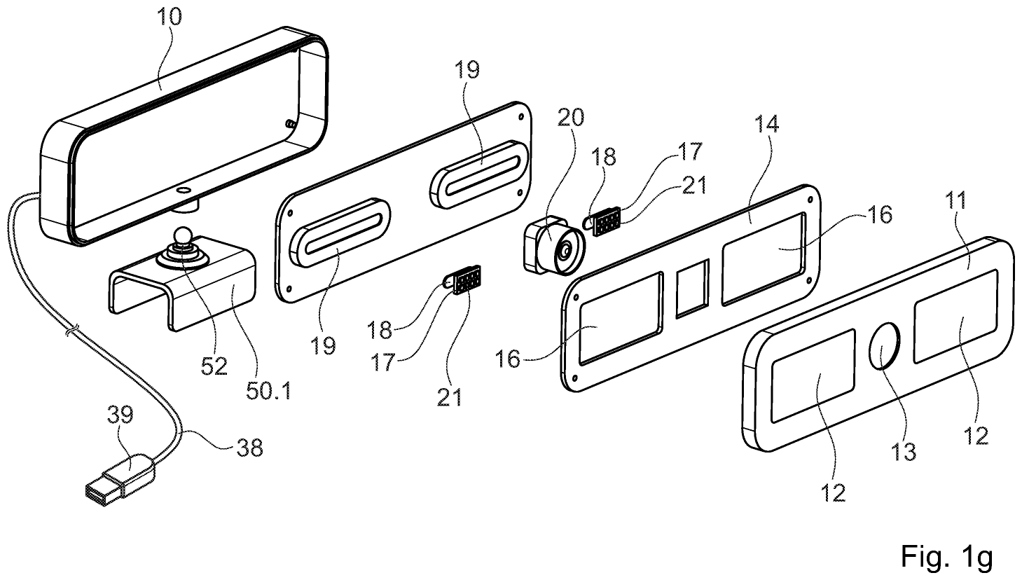

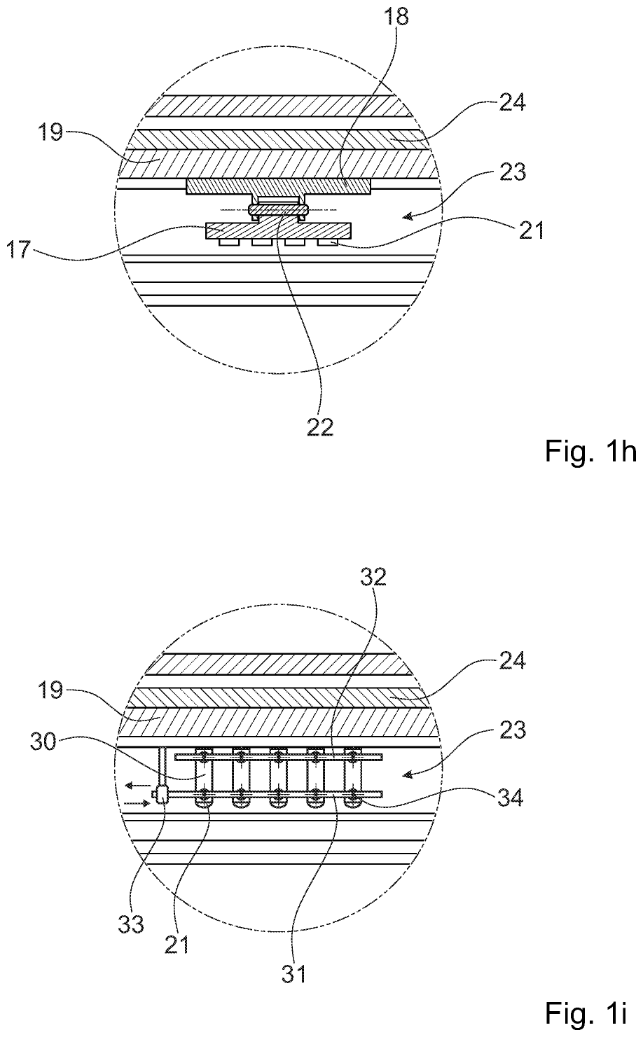

FIG. 1a shows a device 1 according to the invention for stimulating the biosynthesis of vitamin D3. The device 1 is suitable, for example, to be used in the private sector and there to be a supplement to a healthy and conscious lifestyle. The device 1 comprises a housing 10. In the present example, the housing 10 is made of aluminum using a deep drawing process. Of course, housings made of plastic or stainless steel or ceramics are also suitable. A particular advantage of aluminum is its good thermal conductivity, which can help to dissipate any waste heat that may be produced to the environment. The housing 10 is constructed in the form of a shell and is delimited on a front side by a front panel 11. The front panel 11 has two glass windows 12 through which light radiation can penetrate from the inside of the housing 10 to the outside. Although a glass window 12 is discussed at this point, the viewing window shown can also be made of a plastic, for example plexiglass. Ideally, the glass is selected to be substantially transparent to UV-B radiation in the wavelengths of 280 nm to 315 nm. A suitable UV-permeable glass is shown, for example, in U.S. Pat. No. 5,474,589 A (Sumitomo Electric Industries Ltd.).

The light source can be seen through the glass window 12. In the present example, the light source is composed of an array 17 which has a plurality of UV LEDs 21. The array 17 is mounted so that it can be displaced horizontally by being mounted in a light source frame via a light source carriage 18. The housing 10 is connected to a base 41 via a stand 52 connected to the housing 10 with a bearing joint. For attachment to an edge, the base 41 has a gripper, comprising a first gripper 50.1 and a second gripper 50.2, which close a holding groove running centrally between first and second grippers 50.1, 50.2. First and second grippers 50.1, 50.2 are preferably rubberized so that there is additional friction and the grasp of the gripper is improved. The device 1 shown has a power connection via a cable connection 38 which ends in a USB/power plug 39. In the middle of the front panel 11 is the camera lens 13, behind which a camera is installed as an optical sensor.

The device 1 shown in FIG. 1a can be placed, for example, on an upper edge of a computer screen, a television or a tripod device, and a basic adjustment of the alignment of the device 1 can be carried out via the stand 52. The camera lens 13 can monitor a front area and detect any irradiation areas therein. If an irradiation area is identified by a control unit (not shown in FIG. 1a) and the irradiation area is not subject to a proviso, that is to say the irradiation area is not an irradiation area excluded from irradiation, then light radiation is emitted via the light source, in the present example by the UV LED 21. For this purpose, the LEDs 21 can have appropriate reflectors, which bundle the light radiation and correspondingly limit it to the irradiation area (not shown in FIG. 1a).

In operation, the device 1 can exchange numerous data with a corresponding computer. The USB/power plug 39 can be used to monitor the operating parameters, such as, for example, operating time, temperature, irradiation time for individual irradiation areas, and how often a specific irradiation area had to be left out (e. g. the face).

The camera behind the camera lens 13 has a resolution sufficient to enable the control unit to recognize a face. In the simplest implementation of the present invention, the device 1 is only designed to recognize a face as such in order to leave it out from irradiation, or to suspend the radiation if a face is in the irradiation area.

In the present example, it is crucial for the LED that a peak of the emitted wavelength spectrum is in the range of 280 nm to 315 nm. In the present example, an LED with a peak of 298 nm was used. Such LEDs are available from Dowa Electronic Materials Co., Ltd., (Japan), among others.

With this configuration, it was found that a total daily irradiation time of 13 to 14 minutes at a dose of 12.5 mJ/cm² with an erythemal irradiance of 0.015 mW/cm² may be suitable to stimulate and supplement the body's own biosynthesis of vitamin D3 in a skin type 2 user.

In FIG. 1b, the device 1 of FIG. 1a is shown again in a frontal view, only the front panel 11 with the corresponding glass windows 12 and the camera lens 13 being visible from the front. The stand 52 and the first gripper 50.1 formed on the base 41 are also clearly visible. FIG. 1b serves to define the sectional planes used in the figures discussed in detail below, detailing one longitudinal cross section C-C, and two profile cross sections A-A and B-B.

FIG. 1c shows the mentioned longitudinal cross section C-C of FIG. 1b in plan view, i.e. from above onto the device 1. The cross section C-C runs through the center of the height extension of the device 1 and thus through the center of the camera and the lens 13, which, in the present case, are accommodated in the camera compartment of the housing 10. The camera compartment 25 is closed off by a camera lens 26 which is designed flush with the face of the front panel 11. A standard camera, which is suitable for capturing an optical image of the surroundings, can be placed in the camera compartment 25. In this case, the resolution of the camera should be chosen so that it can be evaluated by a control unit and face recognition is possible. The camera lens 26 can be chosen in such a way that it captures a wide angle, for example by a corresponding curvature or cut in the lens increasing the captured area. Such cameras are particularly widespread in the field of surveillance and can be selected by a person skilled in the art if required, taking into account the installation size. The camera forms the sensor unit of the device according to the invention. On both sides of the camera compartment 25 there is a light source compartment 23 in which two light sources are accommodated. The light sources comprise a plurality of UV LEDs and are designed to be movable. Overall, the entire light source compartment is designed as a motion space for the LED. The LEDs are attached to an array and can be pivoted via a pivot arm 22, which pivots the array 17 normal to the plane of the paper at an angle of between ±45°. The pivot arm 22 is connected to a light source carriage 18 which is mounted in a corresponding groove of the light source frame so that it can be displaced horizontally. For example, a simple toothed mechanism with a belt drive can be provided for movement.

The camera (not shown) and the two light sources are mounted on a frame plate 24, which can also comprise the electronic leads (not shown in detail). In addition, the frame panel 24 may comprise the controller. The frame plate 24 can essentially or from individual parts function as a heat exchanger and dissipate any waste heat generated by the light sources to the housing and heat exchanger elements, such as ventilation slots and/or fins, correspondingly formed on the housing. The base 41 can also be seen in plan view. In operation, the light source would be operated synchronously with one another or independently of one another, for example in order to focus on a specific area. In principle, the light sources also comprise reflectors, or the glass windows 12 are configured with appropriate lenses in order to essentially collect the light radiation through the light sources, so that irradiation areas that are as defined as possible are created. The device can be formed to use the camera image to determine whether a person is at a suitable distance from the device in order to decide on the irradiation.

The exemplary device is preferably placeable in a bathroom area where, for example, there are areas of exposed skin as the irradiation areas. A suitable mounting arrangement is, for example, above the bathroom mirror. Reliable face recognition prevents harmful UV-B radiation from being directed at the eyes despite this location.

FIG. 1d shows the device in a side view, the housing 10 being closed off at the face by the front panel 11 and the resulting housing combination being connected to a base 41 via a stand 52. The two grippers 50.1, 50.2 enclose a retaining groove 51.

In a special embodiment, the grippers 50.1, 50.2 are made of a flexible, elastic material so that they exert a restoring force on an object in the retaining groove 51.

FIG. 1e shows the cross section through the center of the device in the plane A-A. In addition or as an alternative to the mounting via flexible grippers 50.1, 50.2 shown in FIG. 1d, this embodiment includes a magnet 53 which additionally enables securing the device 1 at a metallic and/or magnetic retaining element within retaining groove 51. The entire mounting is connected to the housing 10 via a ball joint 55. A frame plate 24 is provided in the housing 10, which carries a chamber area for a camera compartment 25 in the middle, which is closed off at the face by a camera lens 26. The end face is also closed off by the front panel 11.

FIG. 1f shows a corresponding cross section through the axis B-B, in which the light sources are accommodated. The stand 42 is shown here in side view. The light sources are located in a light source compartment 23. If required, small mini fans can be provided in order to supply the light source compartment 23 with a flow of air. These mini fans can carry the waste heat from the light source compartment 23 to the outside via appropriate ventilation slots in the housing 10 and, for example, cool the light source during high-powered operation (not shown). Attached to the frame plate is a light source frame 19, which has a rail in which an array 17 with several UV LEDs 21, light sources that can be displaced normal to the section plane, are attached. Behind the light source frame 19, between the light source frame 19 and the frame plate 24, there is a control compartment 27. The control compartment accommodates the cable guides for the power supply of the light source carriage, the pivot arm 22, the sources and any additional electronics.

FIG. 1g shows the device of FIG. 1a in an exploded view in order to once again clearly define the individual elements. A front panel 11 closes off the face of the device and has several recesses 12,13. A central recess 13 serves to accommodate a camera lens, or allows a camera 20 to capture an image of the area in front of the device 1. On both sides of the camera lens 13 there are two glass windows 12 which are UV-B radiation permeable. A carrier plate 14 defines a light source compartment 23 by two carrier plate recesses 16 and is located directly behind the glass windows 12 when viewed from front to back. Two light sources, comprising LED 21, array 17 and lamp carriage 18, are arranged on both sides of a camera 20 and are accommodated in a light source frame 19. The light source frame 19 is attached to a frame plate 24. The components are accommodated in a housing 10 which is connected to a base via a ball joint which is connected via two grippers 50.1, 50.2. From the housing 10, a communication element or a power supply runs from the inside of the device to the outside via a cable connection 38 with a USB/power plug 39.

FIG. 1h shows in detail the arrangement of a light source in an irradiation room 23. The light source comprises a plurality of LEDs 21 which are designed to emit UV-B radiation. The UVBs mentioned preferably have a peak in a range of 290 to 298 nm. The LEDs are attached to an array 17 which can be pivoted at an angle of +45° via a pivot arm 22. The pivot arm 22 is mounted on a light source carriage, which in turn is movably mounted in a light source frame 19. The electronics of the device according to the invention is designed to move the pivot arm and the light source carriage independently, so that a broad spectrum of irradiation areas can be covered.

FIG. 1h does not show individual reflectors with each LED having a reflector, so that the total radiation from the light source can specifically capture and define/capture and define a specific irradiation area.

An alternative to the construction shown in FIG. 1h can be seen in FIG. 1i. In FIG. 1i, too, the light sources extend into the light source compartment 23. In contrast to FIG. 1h, the individual LED 21 are connected to a light source body which is pivotable on two axes by means of a drive strip 31 and a rear strip 32. A drive strip motor 33 moves the drive strip back and forth in the direction of the arrow while the rear strip is also moved accordingly. This allows the light source bodies to be aligned. If such an arrangement is also provided in the horizontal direction with a corresponding drive strip and a corresponding drive strip motor, the LEDs can thus be pivoted in such a way that a wide cone thereof can be detected by a light beam.

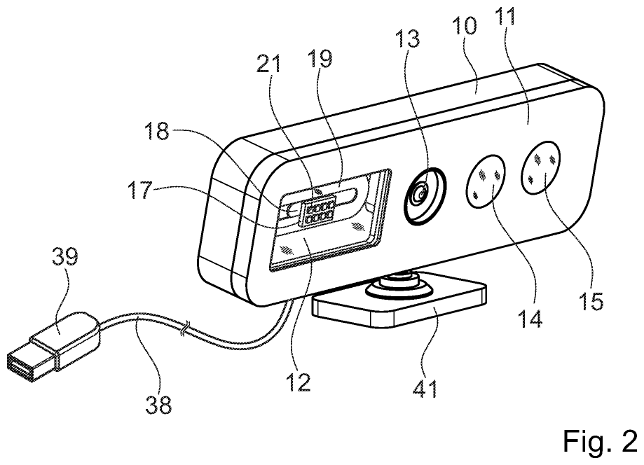

An alternative embodiment of the device is shown in FIG. 2. The device in FIG. 2 essentially has the same structure as that in device 1, but the device in FIG. 2 has additional light sources, namely an additional second light source and an additional third light source. These are provided by glass panes: a first glass pane for the second light source (14) and a second glass pane for the third light source (15). These are provided as a recess in a front panel which, as in FIG. 1, also a camera lens 13 and a glass window 12 with a light source behind it, analogously to how it was described in FIG. 1. This light source is a UV LED diode 21 on an array 17 which is arranged to be movable, namely via a rail which can be traversed by a light source carriage 18 on a light source frame 19. The device can be connected to a power supply or a computer by means of a USB/power plug 39, and thus enable data exchange. As already described in the general section, a device according to the invention can of course also comprise a module for wireless communication, such as, for example, a Wi-Fi or Bluetooth module. Depending on the configuration, the control can take place more or less directly in the device according to the invention. Thus, the device can comprise a processor, which takes over the control tasks, as they are described in the general section, and the execution of the computer program product. It is also conceivable that a third-party computer, e. g. a computer connected via the USB port, takes on this work.

Returning to the example of FIG. 2, where, in addition to the UV-B light source, a second light source with visible light is provided, and a third light source with light in the near infrared range, which is not visible but can improve blood circulation in the irradiated tissue. In addition to having a positive psychosomatic effect, for example by emitting light in a pleasant light color, the light in the visible spectrum can also indicate the operation of the device. Preferably, said first and second light sources are also LED light sources, which have a peak or a spectrum in the wavelength ranges of 380 nm to 720 nm for the second light source, and 720 nm and 1,800 nm, respectively, for the third light source.

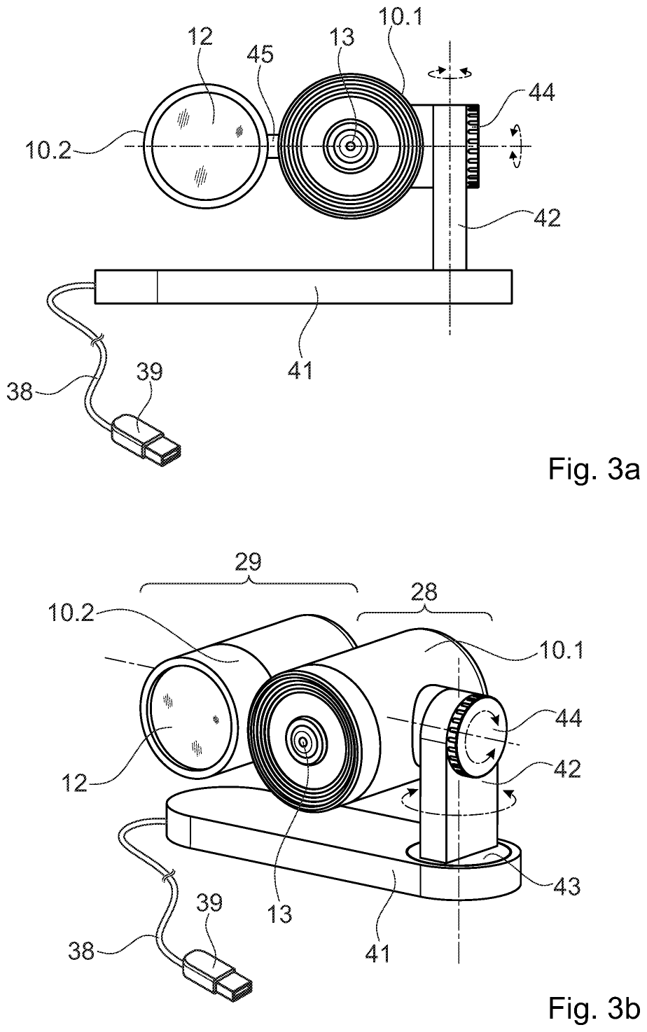

A further alternative embodiment is shown in FIG. 3a. In this embodiment of the present invention, the light source is physically coupled to the sensor unit such that a directing unit moves both synchronously. In other words, the light source is always aligned with the camera area. For this purpose, the device according to the invention has a two-part housing 10.1, 10.2. The first part of the two-part housing 10.1 houses the sensor unit, in the present example a camera with a camera lens 13. The second part of the two-part housing 10.2, however, houses the light source behind a glass window 12. A connection 45 between the first part of the two-part housing 10.1 and the second part of the two-part housing 10.2 is rigid and makes it possible to move both elements simultaneously by means of a directing unit. In this embodiment, the directing unit comprises a rotatably mounted tool arm 44 and a rotatably mounted stand base 43. A stand extends in between. As a result, the device according to the invention can be aligned in a hemispherical area around a base 41 so that overall a very wide effective area can be covered by a lighting element 29 and a camera element 28.

This embodiment also draws its energy from a USB/power connector 39.

The device according to the invention is equipped with a control unit. In all the examples mentioned, the control unit is designed in such a way that it is able to identify the irradiation area. The corresponding sensor units, that is to say the camera elements in the examples shown, recognize an irradiation area and deliver an image to the control unit. Said control unit is designed to identify an irradiation area, namely to evaluate the detected irradiation area and to determine whether this irradiation area is an irradiation area excluded from the irradiation. Whether an irradiation area is excluded from the irradiation can be determined using a predefined database, which is stored, for example, in a memory unit (not shown in the figures). It is also conceivable that the computer program product that takes on the control is capable of determining, by means of intelligent programming based on predefined parameters, whether the irradiation area is such an excluded irradiation area. Certain parameters can be provided for this purpose, such as, for example, the shape and arrangement of recognizable patterns in the captured image area and assignment of these recognizable patterns to certain elements in an irradiation area that is excluded from the radiation. Typically, these are elements utilized for face recognition, such as, for example, the position and distance of the eyes, nose, and mouth to recognize a human face. It is also conceivable that the device shown can connect to a machine-learning-supported program. The computer program product would compare the detected irradiation areas with a database, or use machine learning to determine whether these irradiation areas are possibly excluded irradiation areas. In addition, the computer program product is capable of controlling the irradiation as a whole. This can be done, for example, by the computer program product being designed to assign the duration of an irradiation to a specific irradiation area. In this way it can be ensured that the irradiation area is not exposed twice to the same radiation intensity.

It is to be understood that the device according to the invention requires a control unit for this purpose, which is designed to operate the named computer program product and which is able to execute the corresponding instructions. For this purpose, the control unit can provide a corresponding processor, as well as optional further elements, such as, for example, a computing memory, a main memory and/or an output unit. The output unit can be a simple display, for example. The display can facilitate initial placement of the device according to the invention. These elements can be completely or partially outsourced to a third-party device, namely they do not necessarily have to be located within the housing of the device described above. The computer program product can also be designed to synchronize a plurality of devices according to the invention with one another. The computer program product can thus be designed, for example, to interconnect a plurality of devices arranged in a specific area, such as, for example, a building, with one another.

This can go so far that it can be ensured by face recognition, which is able to assign the face to a specific person, that the person inside a building is exposed to the appropriate dose of light radiation and the appropriate safety measures, such as, for example, that a certain skin area can never be used twice as an irradiation area, can be ensured.

Ideally, a device as shown in this invention, can be used in the private sector. However, the device is particularly suitable in an area in which, for example, skin exposure is greater than in a public space, where usually more clothing is worn. For example, bathing establishments, swimming pools, wellness areas, private bathrooms or cloakrooms are ideal areas in which a device according to the invention, as described here, can be placed. Through the teachings of the present invention, it is possible to make the stimulation of vitamin D3 biosynthesis a casual process, allowing for good compliance by users. Thus, there is no longer a need to take vitamin D3 preparations, which can sometimes be forgotten, or to plan certain rigorous stays outside. In addition to the notorious latitudes, suitable applications include areas, for example, where, due to the circumstances, little natural sunlight shines in during the day, e.g. in underground facilities, large storage and factory complexes, open-plan offices, offshore systems, as well as the interior of buildings in narrow and little-lit side and mountain valleys.

### LIST OF REFERENCE NUMERALS

1) device for stimulating the biosynthesis of vitamin D₃
10) housing
10.1) first part of two-part housing
10.2) second part of two-part housing
11) front panel
12) glass window
13) camera lens
14) glass pane of second light source
15) glass pane of third light source
16) carrier plate recesses
17) arrays
18) light source carriage
19) light source frame
20) camera
21) UV LED diode
22) pivot arm
23) light source compartment
24) frame plate (heat exchanger)
25) camera compartment
26) camera lens
27) control compartment
28) camera element
29) light source element
30) light source body
31) drive strip
32) rear strip
33) drive strip motor
34) pivot joint
38) cable connection
39) USB/power plug
41) base
42) stand
43) rotatably mounted stand base
44) rotatable tool arm
45) connection
50.1) first gripper
50.2) second gripper
51) retaining groove
52) stand
53) magnet
55) ball joint

1. A device for stimulating the biosynthesis of vitamin D₃, comprising:
a. at least one first light source for emitting light radiation comprising wavelengths in a range of between 280 and 315 nm;
b. a sensor unit for detecting an irradiation area;
c. a directing unit for aligning the at least one light source with the irradiation area, and
d. a control unit, which is designed to identify the irradiation area and to trigger an emission of light radiation comprising wavelengths in a range of between 280 and 315 nm by the at least one light source.

2. The device according to claim 1, wherein the sensor unit comprises an optical sensor, in particular a camera.

3. The device according to any one of claim 1 or 2, wherein the control unit is designed to control an alignment of the first light source by the directing unit based on an identified irradiation area.

4. The device according to any one of claims 1 to 3, comprising a collecting unit for generating a directed beam of rays from the emission of light radiation by the first light source.

5. The device according to claim 4, wherein the collecting unit comprises a lens and/or a collimator, in particular comprising a collimator with a collecting lens for generating a directed bundle of rays from the emission of light radiation by the first light source.

6. The device according to any one of claims 1 to 5, comprising a second light source for emitting light radiation comprising wavelengths in the visible range, in particular comprising wavelengths in a range of 380 nm to 720 nm.

7. The device according to anyone of claims 1 to 6, comprising a third light source for emitting light radiation comprising wavelengths in the near infrared range, in particular in a range of 720 nm to 1800 nm.

8. The device according to any one of claims 1 to 7, wherein the control unit is designed to identify a human face in an irradiation area.

9. The device according to any one of claims 1 to 9, wherein the control unit is designed to identify a predefined irradiation area.

10. The device according to any one of claims 1 to 9, comprising a communication unit for exchanging data.

11. The device according to any one of claims 1 to 10, wherein the first light source and/or the second light source and/or the third light source and the sensor unit are physically coupled so that they can be jointly directed at the irradiation area by the directing unit.

12. The device according to any one of the claims, wherein the directing unit is designed to be pivotable in at least two axes, in particular at least one rotatably mounted stand base and a rotatably mounted tool arm for pivoting a light source on at least two axes.

13. The device according to any one of claims 1 to 12, wherein the irradiation area is a skin area with an area of 40 cm² to 900 cm², in particular 50 cm² to 600 cm², in particular of about 400 cm².

14. The device according to claim 13, wherein the skin area of the irradiation area is definable by an adjustable collecting unit.

15. A computer program product for carrying out a control of a device for stimulating the biosynthesis of vitamin D₃, in particular a device according to any one of claims 1 to 13, wherein
a. the computer program product is designed to compare an irradiation area detected by a sensor unit with predefined irradiation areas, and
b. the computer program product is designed to trigger an emission of light radiation comprising wavelengths in a range of 280 nm to 315 nm by at least one first light source if the detected irradiation area does not correspond to an irradiation area excluded from irradiation.

16. The computer program product according to claim 15, wherein an irradiation area excluded from irradiation is an irradiation area selected from the group consisting of: a human face, a skin area provided with one or more pigment nevi, a skin area provided with scab, wound and/or scar tissue, and a skin area already irradiated within a predefined time interval with light radiation comprising wavelengths in a range of 280 nm to 315 nm.

17. The computer program product according to any one of claim 15 or 16, wherein the computer program product is designed to assign a time interval to a specific irradiation area in which the irradiation area is to be irradiated with light radiation comprising wavelengths in a range of 280 nm to 315 nm.

18. The computer program product according to any one of claims 15 to 17, wherein the computer program product is designed to control a search of a predefined irradiation area by the computer program product controlling the sensor unit.

19. The computer program product according to any one of claims 15 to 18, wherein the computer program product is designed to compare each image detected by an optical sensor with an irradiation area excluded from irradiation.

20. The computer program product according to any one of claims 15 to 19, wherein the computer program product is designed to enter an operating mode by a signal from the sensor unit, in particular to search for a suitable irradiation area that is not excluded from irradiation.

21. The computer program product according to any one of claims 15 to 20, wherein the computer program product causes an exposure of an irradiation area to light radiation comprising wavelengths in a range of 280 nm to 315 nm as soon as a sensor unit detects an irradiation area excluded from irradiation in the effective range of the light source.

22. The computer program product according to any one of claims 15 to 21, further designed to monitor or configure the execution of a control of a device for stimulating the biosynthesis of vitamin D₃ on a third-party apparatus via a communication unit.

23. A computer program product which can be executed on a third-party apparatus and is designed to manage user data for a device according to any one of claims 1 to 14,
a. wherein the user data comprises data selected from the group consisting of: skin type, age, occupation and lifestyle, and
b. wherein the computer program product is designed to exchange said user data in encrypted form with a communication unit of the device.
